(19) United States
(12) Patent Application Publication
(10) Pub. No.: US 2006/0084114 A1
(43) Pub. Date: Apr. 20, 2006

(54) Method for detecting cardiac collateral formation

(76) Inventors: Kiang-Tech Jerry Yeo, Etna, NH (US); Michael Simons, Hanover, NH (US)

Correspondence Address: Jane Massey Licata;Licata & Tyrrell P.C., 66 E. Main Street, Marlton, NJ, 08053, US

(21) Appl. No.: 10/966,180

(22) Filed: Oct. 15, 2004

(51) Int. Cl. G01N 33/53; G01N 33/00

(52) U.S. Cl. 435/7.1; 436/86

(57) ABSTRACT

The present invention relates to a method of detecting collateral artery formation by measuring the level of NT-pro-BNP or BNP in an individual.

### BACKGROUND OF THE INVENTION

Coronary collateral circulation has been recognized as an alternative source of blood supply to an ischemic myocardial area. Collateral artery formation is associated with smaller infarcts, less ventricular aneurysm formation, improved ventricular function, fewer future cardiovascular events (Billinger, et al. (2002) J. Am. Coll. Cardiol. 40:1545-50), and improved survival (Hansen (1989) Am. Heart J. 117:290-5).

When blood flow through coronary arteries is insufficient, cardiac ischemia occurs. In response, the body's natural healing process is initiated and the heart may develop limited collateral circulation in an effort to restore blood flow; however, the extent of natural collateral vessel formation in the heart is often inadequate to provide full restoration of blood flow. Thus, therapeutic angiogenesis/artheriogenesis are strategies which have been used to revascularize ischemic myocardial tissue by promoting collateral formation. However, methods for identifying individuals in need of therapy or effectiveness of revascularization are limiting.

### SUMMARY OF THE INVENTION

The present invention is a method for detecting collateral artery formation in an individual. The method involves measuring the level of N-terminal probrain natriuretic peptide (NT-pro-BNP) or brain natriuretic peptide (BNP) in a sample isolated from an individual and comparing said level to a control, wherein an increase in the level in the sample as compared to the control is indicative of collateral artery formation in the individual.

### DETAILED DESCRIPTION OF THE INVENTION

The process of collateral artery formation is not well-defined, although monocytes appear to play a major role. Using angiogenic proteomic profiling of plasma from patents with chronic coronary artery disease (CAD) who further exhibit angiographically visible collaterals, it has now been shown that a strong positive correlation exists between increases in the presence or formation of collateral arteries and NT-pro-BNP levels. Therefore, measuring NT-pro-BNP or BNP levels is useful for monitoring the effectiveness of angiogenic therapy and identifying individuals in need thereof.

Coronary angiography was performed in 96 patients with stable angina pectoris. Plasma levels of NT-pro-BNP was determined in the 96 patients using the commercial Roche ELECSYS® NT-proBNP electrochemiluminescence assay.

Of the 96 patients, 36 patients (42%) had CAD with collaterals (i.e., a collateral score of 2), while 56 patients (58%) had CAD without collaterals (i.e., a collateral score of 0). By multivariate logistic analysis, the presence of collaterals correlated strongly with the angiographic extent of CAD (P=0.014; 95% confidence interval 1.18-4.56). Compared to the CAD without collaterals group, the CAD with collaterals group had significantly elevated median levels of NT-pro-BNP (174 pg/mL vs. 338 pg/mL, p=0.007); a 1.94-fold higher level for the cohort with collaterals.

The findings provided herein indicate that NT-pro-BNP and its processed product, BNP, are useful surrogate markers for collateral formation. Accordingly, the present invention is a method for detecting the presence or formation of cardiac collateral arteries in an individual. The method involves measuring the level of NT-pro-BNP or BNP in a sample isolated from an individual and comparing said level to a control or standard, wherein an increase in the level in the sample as compared to the level of NT-pro-BNP or BNP in the control is indicative of cardiac collateral arteries in the individual.

To measure the level of NT-pro-BNP or BNP, a sample is isolated from an individual. An individual can be, e.g., a patient having, at risk of having, or suspected of having poor collateral circulation; or a patient being given angiogenic therapy, wherein collateral circulation is being monitored. A patient having poor collateral circulation is, e.g., an individual who may have recently been diagnosed with coronary artery disease and may benefit from angiogenic therapy. A patient at risk of having or suspected of having poor collateral circulation is, e.g., an individual who is genetically or physically predisposed to have poor collateral circulation. Further, detection of collateral formation can be in both symptomatic and asymptomatic individuals.

The sample can be a bodily fluid such as whole blood, plasma, serum, or the like, or can be a biopsy sample, isolated according to standard clinical methods. As NT-pro-BNP and BNP are stable in whole blood or plasma at room temperature, special handling of the sample is not required. Further, EDTA and protease inhibitors (e.g., aprotinin) may or may not be added to the sample after isolation to inhibit degradation.

The levels of NT-pro-BNP or BNP are measured using methods provided herein or other suitable assays well-established in the art including, but not limited to, immunoassays (e.g., RIA or EIA); noncompetitive immunoassays, or two-site (sandwich) immunometric assays using two specific monoclonal antibodies or antisera prepared against two sterically remote epitopes of the NT-pro-BNP or BNP chain, and the like.

In accordance with the method of the present invention, a control or standard is used as a reference for identifying increases in NT-pro-BNP or BNP. A control can be the median level of NT-pro-BNP or BNP present in a group of patients (e.g., having CAD) without collaterals. Alternatively, a control can be the level of NT-pro-BNP or BNP in a first sample isolated from an individual before the individual has started a therapeutic angiogenesis regimen (i.e., baseline). Accordingly, in the latter case, the levels of NT-pro-BNP or BNP in the first sample (i.e., the control) are compared to the levels of NT-pro-BNP or BNP in a second sample isolated from the same individual after the individual has started or completed therapy to determine the effectiveness of the therapeutic regimen. It is contemplated that the increase can be expressed as either an absolute increase or percent increase in the levels of NT-pro-BNP or BNP in the sample over control levels.

The method of the present invention can be used alone or in combination with other well-known methods for detecting collateral formation, e.g., coronary angiography, pressure or Doppler sensor-tipped angioplasty, and the like, for selecting patients that might benefit most from an angiogenic treatment regimen or in identifying whether patients are responding to angiogenic therapy to promote vessel growth in the treatment of coronary artery disease.

1. A method for detecting cardiac collateral artery formation in an individual with chronic coronary artery disease comprising measuring the level of an N-terminal probrain natriuretic peptide or brain natriuretic peptide in a first sample isolated from a first individual suspected of having chronic coronary artery disease and comparing said level to a second sample isolated from a second individual known to be free of coronary artery disease, wherein an increase in the level of the N-terminal probrain natriuretic peptide or brain natriuretic peptide in the first sample as compared to the level in the second sample is indicative of collateral artery formation in the first individual.
